(19) United States
(12) Patent Application Publication
(10) Pub. No.: US 2008/0260653 A1
(43) Pub. Date: Oct. 23, 2008

(54) Transdermal Delivery Systems and Transdermal Chelation Preparations

(76) Inventors: Rashid A. Buttar, Huntersville, NC (US); Dean Charles Viktora, Charlotte, NC (US)

Correspondence Address: MORRISON & FOERSTER LLP, 12531 HIGH BLUFF DRIVE, SUITE 100, SAN DIEGO, CA, 92130-2040, US

(21) Appl. No.: 11/568,768

(22) PCT Filed: May 6, 2005
(86) PCT No.: PCT/US05/15871

§ 371 Date: May 12, 2008

Related U.S. Application Data
(60) Provisional application No. 60/569,148, filed on May 6, 2004.

(51) Int. Cl. A61K 9/12; A61K 38/02; A61K 31/00

(52) U.S. Cl. 424/45; 514/2; 424/94.1

(57) ABSTRACT

The invention provides topical chelating preparations and formulations. The invention provides methods of transepithelial delivery of a topical chelating preparation to a human or other animal by topical application to the skin of a human or animal of a topical chelating preparation. In one aspect, a preparation or formulation of the invention comprises a combination comprising of 2-3, dimercaptopropane-1-sulfonate (DMPS) or glutathione, and methionine, in a stabilizing base.

### FIELD OF THE INVENTION

This invention relates to preparations and formulation comprising chelating agents that are serviceable for the detoxification of heavy metals, toxins, poisons, contaminants and other chemicals that are deleterious to health in humans and animals and that can, in non-limiting fashion, be administrated topically or transdermally (transepithelially). Thus, the invention provides preparations and formulations and methods of using them for treating, ameliorating or preventing diseases, conditions, and ailments that can be treated, ameliorated or prevented by the removal/detoxification of heavy metals, toxins, poisons, contaminants and other chemicals that are deleterious to health in humans and animals.

The invention also relates to transdermal (transepithelial) delivery of active agents, chelators, pharmaceuticals, vitamins A, C, E, K, D1, D2, D3, B1, B2, B3, B5 (pantothenate), B6, B7, B9, B12, vitamin H (biotin), beta-carotene, folic acid, niacin, biotin, choline, and co-enzymes (coenzyme Q or Q10, or ubiquinone, acyl-coenzyme A thioesterase, and the like) across the epidermal (skin) barrier of humans and mammals. In one aspect, the invention provides methods for developing new transdermal (transepithelial) delivery systems, e.g., for any polar or non-polar active agent of small or large molecular size. These delivery systems can rapidly delivering the active agent to a targeted location systemically or locally.

### BACKGROUND

Exposure to toxic metals has become an increasingly recognized source of illness worldwide. Metals such as arsenic, cadmium, mercury, iron, tin, lead and chromium are ubiquitous in the environment and exposure through food and water as well as occupational sources can contribute to a spectrum of diseases. Contaminated ground water and acute cases of heavy-metal poisoning are treated with chelators to remove the heavy metals from the contaminated site or person. For example, therapies to reduce mercury load in an individual include the use of 2,3-dimercaptosuccinic acid (DMSA) and 2,3-dimercato-1-propanesulfonic acid (DMPS). Acute cadmium poisoning treatment has included use of the chelating agents ethylenediaminetetraacetic acid (EDTA) and British anti-Lewisite (BAL). Chelating agents such as calcium disodium ethylenediaminetetraacetate (EDTA), 2,3 dimercaptopropanol (BAL), or D-penicillamine (D-PA) have been used as antidotes for the treatment of acute and chronic metal poisoning. Meso-2,3-dimercaptosuccinic acid (DMSA), sodium 2,3-dimercapto-1-propanesulfonate (DMPS) and sodium 4,5-dihydroxybenzene-1,3-disulfonate (TIRON) have also shown to be effective to prevent against toxicity induced by a number of heavy metals. Desferriferrioxamine B (DFB) has been used for the treatment of iron overload. Dimercaptosuccinic acid and dimercaptopropionic sulfonate are also used in the management of human metal intoxications, such as lead, arsenic and mercury compounds.

The pharmaceutical industry is actively seeking to develop new and improved modes of drug delivery to enhance the effectiveness of particular drugs, including, targeting the drug to the intended site, reducing dosage, and decreasing toxicity. Efforts are underway in molecule stabilization for parenteral applications, extended release modalities for enteral drugs and photo-activated chemotherapeutic molecules, for example. Delivery of medications via transdermal drug delivery systems (e.g., patches) has also seen dramatic developments, see U.S. Pat. Nos. 4,879,275; 3,996,934; and 3,731,683. It is now generally agreed within the industry that chemical modification of the barrier properties of the skin is a safe and effective method to enhance penetration of medicaments. However, chemical barrier modifications have their adverse effects, while naturally occurring membrane porosity augmentation and utilization appear to approximate the physiological and biological systems.

### SUMMARY OF THE INVENTION

The invention provides preparations and formulation comprising chelating agents that are serviceable for the detoxification of heavy metals, toxins, poisons, contaminants and other chemicals that are deleterious to health in humans and animals and that can, in non-limiting fashion, be administrated topically or transdermally (transepithelially).

In one aspect, the invention provides compositions for the rapid transdermal (transepithelial) administration of drugs, medicaments, vitamins, coenzymes and/or natural products or other active agents to humans or other animals which does not require use of a “patch” delivery system.

Another object of the invention is to provide compositions effective for transdermal (transepithelial) delivery of active compounds not previously amenable to this route of administration, particularly for pharmacological agents having molecular weights in excess of about 100 daltons and/or at dosages in excess of 0.10 mg per sq cm per 20 minutes, especially, in excess of about 1 mg per sq cm per 20 minutes.

Still another object of the invention is to provide a standardized solvent/carrier base system which is useful for forming topically applied compositions for transdermal (transepithelial) administration of many different chelators or other medicaments with none or only minimal modification required to achieve a true solution of the medicament and effective, safe, and rapid transmigration of the medicament through intact skin.

Another object of the invention is to provide safe and effective compositions for transdermal (transepithelial) administration of a variety of drugs, medicaments, vitamins, coenzymes and/or natural products and other active agents of low or high molecular weight which allows repetitive applications over very short or long periods of time to the same site on the intact skin without causing immunological reaction by the skin.

It is another object of the invention to provide a safe and effective formula compositions for topical transdermal (transepithelial) application of an active agent by matching the solvent/carrier system for the particular active agent which will allow the agent to transmigrate across the skin barrier with no or minimal immunological response at the site of application and without degrading the interstitial skin composition and structure, and without altering the chemical structure or bioactivity of the active agent.

The invention provides preparations or formulations for topical or transdermal (transepithelial) application comprising at least two members, wherein the at least two members are selected from at least two groups of ingredients as set forth in Table 1, below. For example, in alternative embodiments, the invention provides topical or transdermal (transepithelial) preparations or formulations, and devices or materials comprising them, comprising at least one member from at least two different groups as set forth in Table 1, below, e.g., a member from Group 1 and at least one member from Group 2, 3, 4, 5 or 6; or, a member from Group 2 and at least one member from Group 1, 3, 4, 5 or 6; or, a member from Group 3 and at least one member from Group 1, 2, 4, 5 or 6; or, a member from Group 4 and at least one member from Group 1, 2, 3, 5 or 6; or, a member from Group 5 and at least one member from Group 1, 2, 3, 4 or 6; or, a member from Group 6 and at least one member from Group 1, 2, 3, 4 or 5. In separate embodiments, this invention provides different products that comprise (contain at least) all the combinations and permutations of ingredients selected from members of Table 1.

For example, in one aspect, the preparation or formulation comprises: at least one member selected from the group consisting of at least one solvent; and, at least one metal chelator. In one aspect, the solvent comprises a polar solvent, a non-polar solvent, an inorganic solvent or an organic solvent, e.g., wherein the solvent comprises a water, an alcohol, a mixture of water and one or more alcohols, and dimethyl sulfoxide (DMSO). In one aspect, the at least one metal chelator is selected from the group consisting of: 2,3-dimercaptopropanesulphonate sodium (2,3-dimercato-1-propanesulfonic acid, or DMPS); 2,3-dimercaptosuccinic acid (DMSA); British anti-Lewisite (BAL); analogs of BAL; calcium disodium ethylenediaminetetraacetate (EDTA); ethyleneglycol-bis[beta-aiminoethyl ether]-N,N′-tetra-acetic acid (EGTA); diethylenetriamine-pentaacetic acid (DTPA); dipropylene-triamine (DPTA); triethylenetetraaminehexaacetic acid (TTHA); N-hydroxyethylene-diaminehexaacetic-acid (HEDHA); 1,4,7-triazacyclononane-N,N′,N″-triacetic acid (NOTA); 1,4,7,10-tetraazacyclododecane-N,N′,N″,N′″-tetraacetic acid (DOTA); hydroxyethyl-ethylenediaminetriacetic acid (HEDTA, e.g., an N-(2-hydroxyethyl)-ethylenediaminetriacetic acid); a polyaminopolycarboxylic acid; iminodiacetic acid (IDA); cyclam; penicillamine; dimercaptosuccinic acid; tartrate; thiomalic acid; a crown ether; nitrilotriacetatic acid (NTA); 3,6-dioxaoctanedithioamide; 3,6-dioxaoctanediamide; salicyladoximine; dithio-oxamide; 8-hydroxyquinoline; cupferron; 2,2′-thiobis(ethyl acetoacetate); 2,2′-dipyridyl and derivatives thereof; sodium citrate; and, an oxalate salt; wherein alternatively the oxalate salt comprises a sodium oxalate salt, potassium oxalate salt, ammonium oxalate salt or lithium oxalate salt; alternatively the EDTA comprises disodium, trisodium, tetrasodium, dipotassium, tripotassium, dilithium or diammonium salts of EDTA; alternatively the EDTA comprises the barium, calcium, cobalt, copper, dysprosium, europium, iron, indium, lanthanum, magnesium, manganese, nickel, samarium, strontium or zinc chelates of EDTA.

In one aspect, the at least one alcohol is selected from the group consisting of a methanol, an ethanol, a propanol and an isopropanol. In one aspect, the preparation or formulation comprises: at least one solvent; and, at least one member selected from the group consisting of a sulfur-containing amino acid, a sulfur-containing peptide, a sulfur-containing protein and an antioxidant molecule. In one aspect, the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO). In one aspect, the at least one member selected from the group consisting of a sulfur-containing amino acid, a sulfur-containing peptide, a sulfur-containing protein and an antioxidant molecule comprises a glutathione (GSH), a methionine, a cysteine or a cystine.

In one aspect, the preparation or formulation comprises: at least one solvent; and, a humectant or a thickener. In one aspect, the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO). In one aspect, the humectant or thickener comprises a glycerin, a polyethylene glycol, a polypropylene glycol, a glycol, a colloid710h96, an agar, a carbomer, a hydroscopic substituted cellulose, a starch, a propylene glycol glycerine, a mono and poly glycols or a sorbitol. In one aspect, the preparation or formulation comprises: at least one solvent; and, a surfactant. In one aspect, the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO). In one aspect, the surfactant comprises a MJRY-52 (polyoxyethylene separate), an anionic surfactant, a quaternium cationic surfactant or a nonionic surfactant.

The invention provides preparations and formulations comprising at least one solvent; and, a cream, a lotion, an oil, a wax and a wax comprising at least one phospholipid, lipid or fatty acid; wherein alternatively the at least one phospholipid, lipid or fatty acid comprises a lecithin, a phosphatidylcholine, a phosphatidylserine, a phosphatidylethanolamine, a dilinoleylphosphatidylcholine, a lysolipid, a dipalmitoylphosphatidylcholine, a distearoylphosphatidylcholine, a phosphatidylcholine, a phosphatidic acid, a sphingomyelin, a cholesterol, a cholesterol sulfate, a cholesterol hemisuccinate, a tocopherol hemisuccinate, a phosphatidylethanolamine, a phosphatidylinositol, a ferulic acid, a fatty acid, a palmitic acid, a stearic acid, an oleic acid, a linolenic acid, a linoleic acid, a glycosphingolipid, a glucolipid, a glycolipid, a sulphatide, a lipid comprising sulfonated mono-, di-, oligo- or polysaccharides, a lipid comprising an ether or an ester-linked fatty acid, a triglyceride, a high density lipoprotein, a low density lipoprotein, a polymerized lipid, an animal or vegetable oil, a hydrocarbon oil, an ester oil, a silicone oil, a higher fatty acid, a higher alcohol, a sunscreening agent or a flavor. In one aspect, the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO).

The invention provides preparations and formulations comprising at least one solvent; and, a Lewis acid or Lewis base. In one aspect, the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO). In one aspect, the Lewis acid or Lewis base comprises a citric acid, an acetic acid, a niacin or a nicotinamide.

The invention provides preparations and formulations comprising at least one metal chelator; and, at least one member selected from the group consisting of a sulfur-containing amino acid, a sulfur-containing peptide, a sulfur-containing protein and an antioxidant molecule, wherein alternatively the sulfur-containing amino acid, sulfur-containing peptide, sulfur-containing protein or antioxidant molecule comprises a glutathione (GSH), a methionine, a cysteine or a cystine.

The invention provides preparations and formulations comprising at least one metal chelator; and, a humectant or a thickener. The invention provides preparations and formulations comprising at least one metal chelator; and, a surfactant. The invention provides preparations and formulations comprising at least one metal chelator; and, a cream, a lotion, an oil, a wax and a wax comprising at least one phospholipid, lipid or fatty acid. The invention provides preparations and formulations comprising at least one metal chelator; and, a Lewis acid or Lewis base, wherein alternatively the Lewis acid or Lewis base comprises a citric acid, an acetic acid, a niacin or a nicotinamide. In one aspect, the at least one metal chelator is selected from the group consisting of: 2,3-dimercaptopropanesulphonate sodium (2,3-dimercato-1-propanesulfonic acid, or DMPS); 2,3-dimercaptosuccinic acid (DMSA); British anti-Lewisite (BAL); analogs of BAL; calcium disodium ethylenediaminetetraacetate (EDTA); ethyleneglycol-bis[beta-aminoethyl ether]-N,N′-tetra-acetic acid (EGTA); diethylenetriamine-pentaacetic acid (DTPA); dipropylenetriamine (DPTA); triethylenetetraaminehexaacetic acid (TTHA); N-hydroxyethylenediaminehexaacetic-acid (HEDHA); 1,4,7-triazacyclononane-N,N′,N″-triacetic acid (NOTA); 1,4,7,10-tetraazacyclododecane-N,N′,N″,N′″-tetraacetic acid (DOTA); hydroxyethyl-ethylenediaminetriacetic acid (HEDTA, e.g., an N-(2-hydroxyethyl)-ethylenediaminetriacetic acid); a polyaminopolycarboxylic acid; iminodiacetic acid (IDA); cyclam; penicillamine; dimercaptosuccinic acid; tartrate; thiomalic acid; a crown ether; nitrilotriacetatic acid (NTA); 3,6-dioxaoctanedithioamide; 3,6-dioxaoctanediamide; salicyladoximine; dithio-oxamide; 8-hydroxyquinoline; cupferron; 2,2′-thiobis(ethyl acetoacetate); 2,2′-dipyridyl and derivatives thereof; sodium citrate; and, an oxalate salt; wherein alternatively the oxalate salt comprises a sodium oxalate salt, potassium oxalate salt, ammonium oxalate salt or lithium oxalate salt; alternatively the EDTA comprises disodium, trisodium, tetrasodium, dipotassium, tripotassium, dilithium or diammonium salts of EDTA; alternatively the EDTA comprises the barium, calcium, cobalt, copper, dysprosium, europium, iron, indium, lanthanum, magnesium, manganese, nickel, samarium, strontium or zinc chelates of EDTA.

The invention provides preparations and formulations comprising at least one member selected from the group consisting of a sulfur-containing amino acid, a sulfur-containing peptide, a sulfur-containing protein and an antioxidant molecule; and, a humectant or a thickener. The invention provides preparations and formulations comprising at least one member selected from the group consisting of a sulfur-containing amino acid, a sulfur-containing peptide, a sulfur-containing protein and an antioxidant molecule; and, a surfactant. The invention provides preparations and formulations comprising at least one member selected from the group consisting of a sulfur-containing amino acid, a sulfur-containing peptide, a sulfur-containing protein and an antioxidant molecule; and, a cream, a lotion, an oil, a wax and a wax comprising at least one phospholipid, lipid or fatty acid. The invention provides preparations and formulations comprising at least one member selected from the group consisting of a sulfur-containing amino acid, a sulfur-containing peptide, a sulfur-containing protein and an antioxidant molecule; and, a Lewis acid or Lewis base, wherein alternatively the Lewis acid or Lewis base comprises a citric acid, an acetic acid, a niacin or a nicotinamide. In one aspect, the sulfur-containing amino acid, sulfur-containing peptide, sulfur-containing protein or antioxidant molecule comprises a glutathione (GSH), a methionine, a cysteine or a cystine.

The invention provides preparations and formulations comprising at least one humectant or thickener; and, a surfactant. The invention provides preparations and formulations comprising at least one humectant or thickener; and, a cream, a lotion, an oil, a wax and a wax comprising at least one phospholipid, lipid or fatty acid. The invention provides preparations and formulations comprising at least one humectant or thickener; and, a Lewis acid or Lewis base, wherein alternatively the Lewis acid or Lewis base comprises a citric acid, an acetic acid, a niacin or a nicotinamide. In one aspect, the humectant or thickener comprises a glycerin, a polyethylene glycol, a polypropylene glycol, a glycol, a colloid710h96, an agar agar, a carbomer, a hydroscopic substituted cellulose, a starch, a propylene glycol glycerine, a mono and poly glycols or a sorbitol.

The invention provides preparations and formulations comprising at least one surfactant; and, a cream, a lotion, an oil, a wax and a wax comprising at least one phospholipid, lipid or fatty acid. The invention provides preparations and formulations comprising at least one surfactant; and, a Lewis acid or Lewis base, wherein alternatively the Lewis acid or Lewis base comprises a citric acid, an acetic acid, a niacin or a nicotinamide. In one aspect, the at least one surfactant comprises a MJRY-52 (polyoxyethylene separate), an anionic surfactant, a quaternium cationic surfactant or a nonionic surfactant.

The invention provides preparations and formulations comprising at least a cream, a lotion, an oil, a wax or a wax comprising at least one phospholipid, lipid or fatty acid; and, a Lewis acid or Lewis base. In one aspect, the Lewis acid or Lewis base comprises a citric acid, an acetic acid, a niacin or a nicotinamide.

In one aspect, a preparation or formulation of the invention further comprises a compound that alters intracellular osmotic pressure, wherein alternatively the compound comprises niacin, niacinamide, vitamin B3, B5 or B12 and/or biotin. In one aspect, a preparation or formulation of the invention is formulated as a lotion, a cream, a milk, an emulsion, a powder, a spray, an aerosol, a gel or a patch-associated composition.

In one aspect, a preparation or formulation of the invention further comprises drugs, medicaments, vitamins, coenzymes and/or natural products or a combination thereof. In one aspect, a preparation or formulation of the invention is formulated with a drug, a medicament, a vitamin, a coenzyme or a natural product at an effective therapeutic dose of less than about 1 mg per day, or, alternatively, 2, 3, 4, 5, 6, 7, 8, 9, 10 or more mg per day, or, alternatively, 0.9, 0.8, 0.7, 0.6, 0.5, 0.4, 0.3, 0.2 or 0.1 mg per day, or less. In one aspect, a preparation or formulation of the invention comprises molecules that can modify or ameliorate an immune response, wherein alternatively the altered immune response is an altered humoral or cellular response. In one aspect, the drug, medicament, vitamins, coenzyme or natural product has a molecular weight (MW) less than about 340 daltons. In one aspect, the drug, medicament, vitamin, coenzyme or natural product is formulated such that the drug, medicament or natural product is delivered in amounts less than about 1.0, 2, 3, 4, 5, 6, 7, 8, 9 or 10 or more mg per 24 hours.

The drugs, medicaments, vitamins, coenzymes and/or natural products delivered transdermally or transepithelially by a composition of the invention can comprise any drug, medicament, vitamin, coenzyme and/or natural product or combination thereof. For example, iron chelators, e.g., hydroxamates (e.g., desferrioxamine, DFO), amine carboxylates, catechols, hydroxpyridinones and pyridoxal isonicotinoly hydrazones, or a hexadentate Fe(II) chelator can be delivered by a preparation or formulation of the invention. Methods for making and dosing iron chelators are well known e.g., methods for making and delivering hexadentate Fe(II) chelators can be found, e.g., in U.S. Pat. No. 6,589,966. Preparation or formulation of the invention comprising iron chelators can be used to treat iron overload associated with genetic disorders and transfusion-dependent anemias.

The drugs, medicaments, vitamins, coenzymes and/or natural products delivered transdermally or transepithelially by a composition of the invention can comprise vitamins, for example, Vitamin A or beta-carotene, Vitamin B1 (as Thiamin or Thiamin mononitrate), Vitamin B (as riboflavin), Vitamin B3 (as Niacin), Vitamin B6 (as Pyridoxine or Pyridoxine hydrochloride), Vitamin B9 (Folic Acid), Vitamin B12 (cyanocobalamine or hydroxycobalamine), Vitamin H (biotin), Vitamin C (ascorbic acid), Vitamin D, Vitamin E (as dl-alpha acetate), Vitamin K, dexpanthenol, nicotinamide (niacinamide), tocopheryl, and mixtures thereof. Thus, the invention provides methods and compositions for delivering these compositions transdermally or transepithelially.

The invention provides methods of making a specific transdermal delivery system comprising the steps of: (a) selecting one or more active agents, wherein alternatively the active agent comprises a chelator; (b) determining a transdermal effective dose of the active agent: (c) quantifying of effective dose of the active agent; (d) determining an amount of a solvent to solubilize the effective dose of active agent; and, (e) quantifying amount of solvent to solubilize said effective dose.

The invention provides methods of preventing or ameliorating a disease or condition mediated or caused by a heavy metal comprising the following steps: (a) providing a preparation or formulation of the invention; and (b) administering by topical or transdermal application the preparation or formulation of step (a) in an amount effective to prevent or ameliorate the disease or condition.

The invention provides methods for detoxifying an individual of a heavy metal comprising the following steps: (a) providing a preparation or formulation of the invention; and, (b) administering by topical or transdermal application the preparation or formulation of step (a) in an amount effective to detoxify the individual.

The invention provides lotions, creams, milks, emulsions, powders, sprays, aerosols, gels, patches and the like comprising a preparation or formulation of the invention. In one aspect, the lotion, cream, milk, emulsion, powder, spray, aerosol, gel or patch further comprises a drug, a medicament, vitamin, coenzyme or a natural product. In one aspect, the lotion, cream, milk, emulsion, powder, spray, aerosol, gel or patch is formulated such that the drug, medicament, vitamin, coenzyme or natural product is delivered in amounts less than about 1.0, 2, 3, 4, 5, 6, 7, 8, 9 or 10 or more mg per 24 hours.

The invention provides preparations or formulation comprising a 2-3, dimercaptopropane-1-sulfonate (DMPS), a glutathione and/or a methionine formulated in a stabilizing base, used, e.g., for transdermal or transepithelial delivery of a composition. The invention provides preparations or formulation comprising a 2-3, dimercaptopropane-1-sulfonate (DMPS) or a glutathione, wherein alternatively the preparation or formulation further comprises a methionine.

The invention provides a lotion, a cream, a milk, an emulsion, a powder, a spray, an aerosol, a gel or a patch comprising a 2-3, dimercaptopropane-1-sulfonate (DMPS), a glutathione and/or a methionine. The invention provides a lotion, a cream, a milk, an emulsion, a powder, a spray, an aerosol, a gel or a patch comprising a 2-3, dimercaptopropane-1-sulfonate (DMPS) or a glutathione, wherein alternatively the preparation or formulation further comprises a methionine.

The invention provides preparations or formulations comprising water, DMPS or glutathione (or DMPS and glutathione), glycerin, polyethylene glycol, MJRY50, cream and citric acid, used, e.g., for transdermal or transepithelial delivery of a composition. The invention provides a lotion, a cream, a milk, an emulsion, a powder, a spray, an aerosol, a gel or a patch comprising water, DMPS or glutathione (or DMPS and glutathione), glycerin, polyethylene glycol, MJRY50, cream and citric acid. The invention provides preparations or formulations comprising water, glutathione, glycerin, polyethylene glycol, MJRY50 and a cream. The invention provides a lotion, a cream, a milk, an emulsion, a powder, a spray, an aerosol, a gel or a patch comprising water, glutathione, glycerin, polyethylene glycol or equivalent, a surfactant (e.g., MJRY50) and, alternatively, a cream. The invention provides a lotion or cream comprising lecithin, propylene glycol, a surfactant and a sorbitol, wherein alternatively the surfactant is MyrJ-52. In one aspect, a preparation or formulation of the invention comprises a combination comprising of 2-3, dinercaptopropane-1-sulfonate (DMPS) or glutathione (or DMPS and glutathione), and methionine, in a stabilizing base.

The invention provides a lotion, a cream, a milk, an emulsion, a powder, a spray, an aerosol, a gel or a patch comprising a preparation or formulation of the invention, and Vitamin A or beta-carotene, Vitamin B1 (as Thiamin or Thiamin mononitrate), Vitamin B (as riboflavin), Vitamin B3 (as Niacin), Vitamin B6 (as Pyridoxine or Pyridoxine hydrochloride), Vitamin B9 (Folic Acid), Vitamin B12 (cyanocobalamine or hydroxycobalamine), Vitamin H (biotin), Vitamin C (ascorbic acid), Vitamin D, Vitamin E (as dl-alpha acetate), Vitamin K, dexpanthenol, nicotinamide (niacinamide), tocopheryl, or a mixture thereof.

The details of one or more embodiments of the invention are set forth in the accompanying drawings and the description below. Other features, objects, and advantages of the invention will be apparent from the description and drawings, and from the claims.

All publications, patents and patent applications cited herein are hereby expressly incorporated by reference for all purposes.

### BRIEF DESCRIPTION OF THE DRAWINGS

FIG. 1 is a graphical representation of the results obtained in Example XX, for the flux (.mu.g/cm.sup.2) vs. time (m), of DMPS (as dimercaptopropanesulfonic sodium) under open (lotion) conditions using the topical delivery system.

FIG. 2 is a graphical representation of the results obtained in Example XX, for the flux (.mu.g/cm.sup.2) vs. time (m), of EDTA (as Edetate Disodium) under open (lotion) conditions using the topical delivery system.

### DETAILED DESCRIPTION OF THE INVENTION

This invention provides novel preparations and formulations comprising chelating agents that are serviceable for the detoxification of heavy metals, toxins, poisons, contaminants and other chemicals that are deleterious to health in humans and animals and that can, in non-limiting fashion, be administrated topically or transdermally. This invention provides compositions for rapid and non-irritating transdermal delivery of pharmaceutically active agents and methods for formulating such compositions and delivery thereof.

The invention also relates to transdermal delivery of active agents, chelators, pharmaceuticals and co-enzymes (B1, B2, B3, B5, B6, B7, and the like) across the epidermal (skin) barrier of humans and mammals. In one aspect, the invention provides methods for developing new transdermal delivery systems, e.g., for any polar or non-polar active agent of small or large molecular size. These delivery systems can rapidly delivering the active agent to a targeted location systemically or locally.

In alternative aspects, a trans-dermal delivery formulation or system of the invention is applied as a lotion, cream, milk, emulsion, powder, spray, aerosol or gel. In one aspect, the trans-dermal system comprises a particular active agent dissolved in a stabilizing aqueous solvent system; where the medicament can form a true solution which can rapidly crosses the stratum corneum of the skin. In one aspect, the trans-dermal system comprises a non-aqueous system; which can protect the medicament from environmental degradation during application to the stratum corneum. The system may also comprise one or more “humectants” and/or “cellular respiration inducers”, which indirectly increases the intracellular protein swelling and subsequent increases in epidermal hydrophilic qualities.

In alternative aspects, a trans-dermal delivery formulation or system of the invention is formulated as a single lotion, milk, cream, powder, spray, aerosol or gel based system, which can stabilize and protect the chelator form degradation, permit selective application and delivery of the chelator to the proximus (proximity) of the stratum corneum, assist the chelator to transit the stratum corneum, protect the chelator from environmental degradation by, e.g., developing an over-laying non-aqueous protective layer. In alternative aspects, a trans-dermal delivery formulation or system of the invention is formulated to comprise a primary and/or a secondary agent(s) to positively affect the intracellular osmotic pressure to induce immediate absorption through the cell membrane (stratum corneum) into sub layers. In one aspect, the non-aqueous portion can isolate the aqueous layer against the stratum corneum and maintain the ion gradient across the stratum corneum.

In alternative aspects, a trans-dermal delivery formulation or system of the invention is formulated to comprise a two part solution system comprising an aqueous and non-aqueous phase and/or alternatively as a highly hydrophilic phase and/or a highly hydrophobic phase, respectively.

Aqueous Phase (hydrophilic): In one aspect, a primary stable aqueous solution, which can protect the chelator from degradation during storage, use, and when applied to and transiting the stratum corneum into the granular matrix.

Non Aqueous Phase (hydrophobic): In one aspect, a secondary non-aqueous fragile formulation, which can separate (break) over the aqueous component (adhering to the skin surface), which can cover and/or protect the medicament and area from environmental attack. In one aspect, the non-aqueous portion is designed to stabilize the aqueous ion concentration and to have a secondary and residual release of the humectants form the secondary non-aqueous portion, which maintains the Donnan effect of compartmental (intracellular) swelling, with the associated osmotic uptake of water and chelator through the stratum corneum, and, in one aspect, concomitant increased and continual uptake of chelator, which rapidly passes form high gradient to low ion gradient. While the invention is not limited by any particular mechanism of action or biologic process, the imbalance (direct or indirect) of the intracellular space can optimize the Donnan effect making the stratum corneum (membrane) relatively freely permeable to medicaments and permitting entrance into the granular layer, into the spinous and basal layers of the epithelium.

In one aspect, osmotic pressure is effected by administering niacin, niacinamide, vitamin B3, B5 B12, dinitrophenol, and/or biotin (and the like), to altering osmotic pressure indirectly, e.g., by altering cellular respiration—which can alter intracellular osmotic pressure. Thus, in one aspect, a trans-dermal delivery formulation or system of the invention is formulated to comprise niacin, niacinamide, vitamin B3, B5 or B12, dinitrophenol, and/or biotin, or any equivalent compound that can alter intracellular osmotic pressure.

In alternative aspects, a trans-dermal (transepithelial) delivery preparation, formulation or system of the invention comprises a drug, a medicament, a vitamin, a coenzyme and/or a natural product. In one aspect, the composition of the invention is formulated with a drug or a medicament at an effective therapeutic dose of less than about 1 mg per day, or, alternatively, 2, 3, 4, 5, 6, 7, 8, 9, 10 or more mg per day, or, alternatively, 0.9, 0.8, 0.7, 0.6, 0.5, 0.4, 0.3, 0.2 or 0.1 mg per day, or less. In one aspect, the preparation, formulation or system of the invention comprises molecules which ameliorate modify or an immune response (e.g., humoral or cellular) when transmigrating the skin.

In alternative aspects, a trans-dermal delivery preparation, formulation or system of the invention comprises a drug, a medicament, a vitamin, a coenzyme and/or a natural product having molecular weights (MW) less than about 340 daltons. In alternative aspects, a trans-dermal delivery preparation, formulation or system of the invention are formulated such that a drug, a medicament or natural product in the preparation, formulation or system is delivered in amounts less than about 1.0, 2, 3, 4, 5, 6, 7, 8, 9 or 10 or more mg per 24 hours. In alternative aspects, the drug, medicament, vitamin, coenzyme and/or a natural product is soluble in an alcohol, e.g., an ethanol and/or isopropanol, or a glycol or a dimethyl sulfoxide (DMSO), and, in one aspect is not be chemically altered by solubilization.

A potentially limiting factor for compounds which can have efficacy at relatively small doses when introduced systemically via the capillary net of the dermis is molecule size and irritation potential of the drug, medicament, vitamin, coenzyme and/or a natural product plus solvent(s) and other components. Routine screening tests can determine the appropriate dosages, concentrations and relative proportions of various compounds in a particular preparation, formulation or system of the invention, depending on the desired effect and/or purpose.

Another potentially limiting factor for compounds used in a preparation, formulation or system of the invention in transdermal drug delivery is when drug carriers chemically bind with a drug, a medicament, a vitamin, a coenzyme and/or a natural product, resulting in non-bioavailable compounds transmigrating the skin; or/and the carrier, e.g., DMSO, may reduce the medicament yielding a non-bioavailable or non-bioequivalent compound. Again, routine screening tests can determine the appropriate dosages, concentrations and relative proportions of various compounds in a particular preparation, formulation or system of the invention, depending on the desired effect and/or purpose.

The anatomy and physiology of the integument was analyzed to understand the complex protective mechanism of physical, biochemical and bio-electrical gradients which work to minimize the penetration of foreign substances and sensitize the organism to react more rapidly and aggressively to future exposures. Noting that the invention is not limited by any particular mechanism of action or biologic effect, as result of this analysis it is postulated that the primary pathway of trans-dermally delivered drugs is paracellular, i.e., around the cells, through the intracellular space. The museledge-like compound—composed of elastin, collagen and hyaluronic acid and other lipids, which occupies the interstices between the cells of the top-most layer of the skin (i.e., the epidermis, including, e.g., stratum corneum, lucidum, granulosum, spinosus), need not be dissolved (or otherwise disrupted) in order for a medicament or other active agent, dissolved in a solvent, to transmigrate through viable skin to the subcutaneous tissues where the cutaneous plexi of the capillary net can be reached and/or deeper penetration achieved. The only significant requirement is to utilize and enhance the Donnan effect, which causes membrane and intracellular swelling, which induces immediate hydration and associated enhanced permeability.

Surfactants (surfactive active agents), which delipidize and de-glycolize the stratum corneum can reduce the protective barrier capacity as a function of species and concentration, whereupon sub-cutaneous swelling induces hydration (and associated flux or medicament. Thus, in practicing the compositions and methods of the invention, permeability can be adjusted by modifying the choice of surfactant and associated hydration (HLB). In one aspect, the addition of a second non-aqueous layer is advantageous to hold, restrict, protect the underlying, primary aqueous layer.

Capillary circulation can act as a sink for a medicament, thus maintaining a continual ion potential gradient across the skin. Diffusivity of a drug molecule is dependent on properties of both the medicament and the medium (carrier). The diffusivity in liquid media (in general) tends to decrease with increased polarity, molecular weight and volume. The rate of skin penetration is a function of the Donnan effect, the Diffusion Coefficient, the barrier partitioning tendencies, binding affinities, and the rate of metabolism of the medicament by the skin. The Donnan effect and associated Diffusion Coefficient of the medicament is influenced by molecular weight (MW), molecular structure, solubility, additives, rate of metabolism of the medicament by the skin. Diffusion is also dependent on the carrier, and how the carrier solution and medicament can be held in contact with the stratum corneum. Thus, these factors are considered when practicing the invention and determining the dosages and formulation content of compositions of the invention.

An optimum associated hydration, or “Hydrophilic-Lipophilic Balance” (HLB), is not required for a medicament to penetrate efficiently. An approximate HLB will suffice. A high HLB will not permit the non-aqueous/aqueous portions to “break upon the skin”, leaving a creamy residue, with sequestered medicament. A low HLB will prematurely break, permitting excessive evaporation and adhesion of the medicament to the surface of the stratum corneum and will not form a proper protective non-aqueous film. Thus, HLB is considered when practicing the invention and determining the dosages and formulation content of compositions of the invention. The term “HLB” is an arbitrary scale from 0 to 40 depicting the Hydrophilic/Lipophilic Balance of a surfactant. Products with low HLB are more oil soluble. High HLB represents good water solubility. For example, lipophilic, or nonpolar emulsifiers, have HLB numbers below 9, while hydrophilic (polar) emulsifiers have HLB numbers above 11. Note that HLB is a numerically calculated number based on the surfactants molecular structure. It is not a measured parameter.

Highly lipophilic chelators are very soluble in selected oils and lipids, and will transit the stratum corneum easily. Therefore, highly lipophilic chelators can be easily incorporated into the trans-dermal creams, lotions, oils, gels, and milks of the invention.

Skin may metabolize some drugs (e.g., chelators) effectively. Therefore inhibition and fluxes of medicament/metabolite are important to consider when practicing the invention and determining the dosages and formulation content of compositions of the invention. For example, to what extent the drug metabolizes to a different form and how rapidly and completely does a chelator transit the skin can be considered when practicing the invention.

Ionized species of medicaments transmigrate more readily. Generally, ionized species are several orders of magnitude more permeable than their non-ionized form. Accordingly, in one aspect ionized species of drugs, medicaments and/or natural products are used in the preparations or formulations of the invention.

Transient increases in cutaneous blood flows may result in increased systemic absorption of the drug from the intracellular spaces. In practicing the invention, cellular biological issues can be reviewed in order to identify and categorize membrane and organelle functions, both in the integument and in other tissues, which might be subject to variations which might help or hinder tissue transmigration of a desired drug, medicament and/or natural product and solvent.

Solvent and surfactant systems can cause cutaneous allergic responses from the medicament they are delivering. Chronic exposure to irritants has the potential to become pathological and, therefore, consideration must be given in the formulation of the systems. The objective is to have minimal surfactant to penetrate the skin, and to be functionally isolated to the skin surface. Because of the relative high molecular weights of and polarity of the surfactants, it is difficult for them to penetrate the stratum corneum. Individuals might exhibit hepatic toxicity. Thus, the possibility of cutaneous allergic responses is considered when practicing the invention and determining the dosages and formulation content of compositions of the invention, and in practicing the methods of the invention. Screening protocols to determine the possibility of cutaneous allergic responses are well known in the art.

In one aspect, the preparations and formulations of the invention have one or more, or all, of the following characteristics and features: ability to dissolve and emulsify the active agent down to individual molecules (true solutions) in a carrier which remains liquid long enough to penetrate the epidermis; remains stable as formulated and not form an irreversible complex with other substances; does not damage the skin or elicit an immunological response with repeated use; releases the active agent appropriately upon contact with the stratum corneum and does not alter the active agent, or leave as residual compounds which might be sensitizing, and for aqueous soluble compounds, provides a secondary protective non-aqueous protective layer, and for oil soluble chelators, provides a secondary protective aqueous protective layer.

In one aspect, the invention provides a topical formulation for the transdermal delivery of an active agent which addresses the design of the integument as a biologically responsive physical, chemical and bioelectrical barrier against the active agent(s) and solvent(s). In one aspect, solvent and surfactant component(s) used in the invention are selected so that no permanent or strong covalent bonds with the medicament or other active agent are formed, wherein the surfactants alter the surface permeability and are readily stripped (by molecular weight & polar motility versus protein permeability) from the active agent at the intended stratum corneum site of application, thereby liberating the active aqueous associated agent to enter the biological system once released. In one aspect, the transmitted intracellular solution further facilitates movement of the medicament into the granular, spinous, and basal layers, whereupon it can enter the circulatory system of blood, lymphatic system, or biological system(s).

In one aspect, the formulations of the invention are designed to modify the ‘Gel’ characteristics of the epidermis, to cause an un-equilibrium, which produces a significant Donnan Effect, which is associated with rapid hydration and solute influx, thereby facilitating transmigration through the skin. While the invention is not limited by any particular mechanism of action, in one aspect, by facilitating the transmigration and increasing the rate of diffusion of the active agent and other system components through the skin the less time the formulation will have to remain in the tissues and the lower the physiological response. In one aspect, this is accomplished by selecting solvent(s) and modifier(s) to provide a true solution, namely a solution of the various components in the solvent system on a molecular level, while at the same time forming a protective “coating” or temporary complex with the active agent to facilitate its intact transmigration through the skin.

The present invention also provides transdermal drug delivery systems which may include a substance which can assist the skin in repairing damage which is caused by the transmigration of the delivery system. The formulation may include one or more additional ingredients effective for enhancing percutaneous absorption and the stabilization of the active agent in its intact, bioactive form. Such additional agents include, for example, one or more cofactors: Vitamin B sub 1, 2,3, 5, 6, and 7, biotin and/or Panthenol, and thickeners: i.e., sol fiber, acrylates, PVA, etc. The present invention also provides for one or more surfactants, DMSO glycerylmonolaurate, quaternium cationic surfactants, N,N-dialkyl alkanolamines, such as N,N-diethylethanolamine. The non-aqueous portion may also include vitamin A, E, and D sub. 3, 4. and lecithin.

In one aspect, the topical, liquid, composition is effective for transdermal delivery of low and high molecular weight active agent, especially medicaments and other active agents having molecular weights of about 100 D and/or at dosages in excess of 0.10 mg per cm per 20 minutes, especially, in excess of about 1 mg per sq cm per 20 minutes. In one aspect of this embodiment, the composition is formulated as a unit dosage, e.g. one milliliter containing from about 0.001 to about 300 mg of active agent having molecular weight of at least about 100 daltons (D) in a carrier in which the active agent is completely dissolved. In one aspect, the carrier includes a solvent system in which the active agent is at least substantially soluble, at least one solvent modifying compound to facilitate transdermal delivery of the active agent and, as necessary, to increase suspension of non-aqueous agent in the solvent system; and/or at least one solute (active agent) modifying compound forming a non-covalently bonded complex with the solvent.

In one aspect, rapid introduction of the active agent enables minimal immune response or anaphylaxis, and repetitive dosing over the same area of skin over a short term or, if needed, for a longer course of therapy. In order to accomplish this and other objectives, in one aspect, the delivery system is designed to (1) create a transient modification of those aspects of the solvents and solutes which encounter or trigger the body's defense mechanisms against dermal transmigration and, (2) minimize or offset any damage done by dermal transmigration.

In one aspect, the transient modification (1) is manifested by the formation of a complex between the solute (active agent) and the solvent or solvents and modifying agents or modifiers for the solvent(s) and/or the solute. In one aspect, these complexes are formed as non-chemical true solutions of the solute in solvent. In one aspect, the carriers of the transdermal delivery system of this invention are designed for each particular drug or other medicament or active agent which allows the resulting complex of active agent to pass through each of the different layers of the skin's defenses with minimal or no irritation. In one aspect, as the complex passes through each layer or layers one or more modifying agents of the complex may be stripped away from the complex, usually by preferentially bonding or reacting with a component or components of the skin layer, but without reacting or disassociating the active agent. This mechanism can allow the active agent to reach and be absorbed by or react with its intended target, usually absorption into the vascular or capillary network.

In practice, however, in view of the overall similarities of common structural elements with and among large classes of medicaments, a standard or stock solution of the invention which, with only minor modifications or fine tuning, can be used for many different active agents. In one aspect, the stock solution includes (A) solvent(s); modifying agents including (B) solvent modifier(s); and/or (C) metabolizeable solute stabilizer(s); (D) source(s) of cellular activation energy; and/or (E) skin stabilizer(s). Alternative ingredients may also be included, for example, (F) capillary dilator(s). In one aspect, the active agent is mixed with the stock solution, further modified, as necessary, to increase solubility and/or more closely match the molecular properties of the stock solution plus active agent to that of the active agent, taking into account one or more effects of the molecular interactions of molecules in a liquid.

It is understood that all ingredients used in the compositions of this invention must, within the applied and recommended dosages, be non-toxic and safe for human (mammalian) use. Also, all amounts, parts and percentages in the following description of exemplary embodiments and appended claims are on a weight basis unless otherwise noted.

(A) Solvents

In one aspect, the solvent is the principal component of the carrier for the active agent and, preferably, is one in which the active agent is soluble or at least substantially soluble or can be made soluble or become more soluble, by addition of one or more solvent modifying agents. As used herein, by “substantially soluble” is meant that the minimum effective dose of the active agent, generally at least about 0.25 mg, preferably at least about 0.5 mg, especially preferably about 1 mg, or more, will dissolve in 1 cc of the solvent(s) or in 1 cc of a mixture of the solvent(s) with solvent modifying agent(s). Suitable solvents may be selected from any of the solvents normally used for medicaments, cosmetics, nutrients or other active agent to be delivered transdermally.

Exemplary solvents include water, polyols, such as, for example, propylene glycol, butylene glycol, glycerol (s). Mixtures of solvents may be used. Other solvents may also be used, in amounts which will be safe and non-toxic in use. In one aspect, the solvent system is aqueous. In one aspect, other agents may be used for water soluble active agents and for those drugs or other active agents which are stable in the presence of and not denigrated by their presence. In one aspect, when other solvent agents are present, it will usually constitute less than about 20 percent, preferably less than about 10 percent by weight of the total solvent although more or less may be used depending on the active agent and so long as the objective of the invention can be met. In one aspect, the compositions and methods of this invention may be formulated as an oil, cream, milk, lotion, spray, aerosol or gel.

In one aspect, the total amount of solvent(s) will be selected to assure dissolution of the solute and other additives and provide suitable product viscosity. In one aspect, the amount of solvent(s) fall within the range of from about 0.2% to about 99.8 percent.

(B) Degradation Inhibitors

In one aspect, a solvent modifier is selected to modify the polarity of the solvent system to closely match that of the active ingredient (solute). In one aspect, solvent modifiers are polar compounds (from polar ions in solution) and can contain a functional group containing oxygen, sulfur or nitrogen in its molecule. In one aspect, the inhibitor/stabilizer(s) enable a weak complex with the active agent, and/or is the first sacrificial agent to be oxidized or reduced. As used herein, “stabilizer” is intended to have its normal and usual meaning, namely, that the composition may be stored at room or elevated temperature for one or more days, usually 30 or more days, without undergoing significant degradation of the active agent or solution.

In one aspect, inhibitors or stabilizers may be individually (or as a group) selected from substances having structural elements in common with the active agent. However, it has been found that for many bio-active compounds and other active agents, a relatively small group of inhibitors/stabilizers facilitate the stability of the active agent and formation of the weak association which enable the complex of stabilized active agent to pass the defenses of the skin with minimal irritation without modification of the chemical structure or stereoscopic configuration of the active agent. Thus, in alternative aspects, stabilizers of sulfur containing amino acids and amino acid conjugates of two or more, pro-Vitamin C, Vitamin C, tocopherols (sub alpha, beta, gamma, delta), Vitamin B1, 2, 3, 5, 6, 7, Pro-Vitamin B1, 2, 3, 5, 6, and 7, Biotin, Folic acid and pro-folic acid, D-Panthenol, alpha lipoic acid, pro-alpha lipoic acid, and methylsulfonylmethane (MSM) and/or other commercially available inhibitors (substituted phenols) are used in formulating the compositions of the invention.

In one aspect, the amount of inhibitors is selected to result in the desired stability ratio, and will depend on various factors. In one aspect, the suitable amount of solvent modifier(s) to achieve the desired ratio falls within the range of from about 0.0001 to about 150% of the active ingredient(s).

(c) Surfactants and Humectants

A solute modifier may be included in the formulation of the topical delivery system where necessary to facilitate dissolution and to hold the active ingredient(s) in proximity of the stratum corneum. In one aspect, solute modifiers which form reversible and/or temporary complexes with the solute to facilitate passage through the skin while minimizing immunological response are especially effective. The solute modifier, optimally, will not transit the stratum corneum and/or will only transit in minute quantities) to prevent or reduce the possibilities of hepatic toxicity.

Exemplary solute modifiers include, e.g., sulfonate and oxygenated compounds, dialkyloids, glucoside, genistin, polyphenolic, sterol, such as, for example, cholesterol and cholesterol-like compounds and hormones, 3,3′-thiodipropionic acid (sulfurated propionic acid), phosphatidyl serine and choline, lecithin(s), dehydroepiandosterone (DHEA), phosphatidyl serine, phosphatidyl choline, and naturally occurring soaps and sapogins. Examples of humectants are propylene glycol glycerine, mono and poly glycols, sorbitol, and equivalent compounds, e.g., any compound that absorbs or retains moisture, including substances that preserve the moisture or water content of the skin, such as alpha hydroxy acids such as lactic acid.

The selection of the particular humectants will facilitate movement of the solute-complex past the stratum corneum and viable skin, deep in the tissues, to its optimal targeted internal circulation system of blood. The suitable amount of the surfactant or humectant may be determined based on such factors as, for example, solubility of the modifier in the system (e.g. solvent plus solvent modifiers), its molecular compatibility with the solute, its ability to affect increase or decrease concentration (solubility) of solute in the solvent, etc. In alternative aspects, the amount of solute modifier is at least about 0.003%, such as, for example, from about 0.003 to about 50%, or from about 0.1 to about 5%, or from 0.1 to about 4%, based on the weight of total composition.

In alternative aspects, the above described humectants, surfactants, and soluble fiber (thickeners) of this invention, are selected from substances which the body recognizes as usable building blocks or benign (not pathological to the physiological or biological systems). This selection therefore facilitates nearly complete disassociation of the medicament from the delivery system once transiting into the body. Since these carrier/complex compounds are reducible to elemental building blocks of physiology they should do no harm to the body.

(D) Indirect Effects on Intracellular State (Interstitial)

In alternative aspects, the processes by which transdermal drug delivery operate involve moving molecules across chemical and electrical gradients. Alteration of the interstitial space by inducing unregulated cellular respiration alters the protein gel hydrophilic characteristics.

In alternative aspects, substances which produce unregulated cellular respiration are B3, B5, B12, dinitrophenol, and/or biotin are used in formulating compositions of the invention. In alternative aspects, these substances (e.g., B3, B5 or B12, dinitrophenol, and/or biotin) will fall within the range of from about 0.001 to about 0.1%, or, from about 0.001 to about 0.01%, or, from about 0.001 to about 0.005%, or, from about 0.0001 to about 0.005% or, from about 0.00001 to about 0.005%, based on total composition.

As used herein, a “transdermal chelation preparation” as provided herein is abbreviated “TDCP”; likewise transdermal chelation preparations (plural) as provided herein may be abbreviated “TDCPs”.

In separate but non-limiting exemplifications, this invention provides novel transdermal chelation preparations (TDCPs) comprised of: any “N2-1000” (i.e., numbering two to one thousand) members selected from at least two groups of ingredients as shown in Table 1, where “N2-1000” is any integer from 2 up to 1000. Thus—to illustrate—in one exemplification, this invention provides novel TDCPs comprised of: any two members selected from at least two groups of ingredients as shown in Table 1; in another exemplification, this invention provides novel TDCPs comprised of: any three members selected from at least two groups of ingredients as shown in Table 1; in another exemplification, this invention provides novel TDCPs comprised of: any four members selected from at least two groups of ingredients as shown in Table 1; in another exemplification, this invention provides novel TDCPs comprised of: any five members selected from at least two groups of ingredients as shown in Table 1; (and so on).; and in another exemplification, this invention provides novel TDCPs comprised of: any one thousand members selected from at least two groups of ingredients as shown in Table 1.

In separate but non-limiting exemplifications, this invention provides novel TDCPs comprised of: any “N3-1000” members selected from at least three groups of ingredients as shown in Table 1, where “N3-1000” is any integer from 3 up to 1000. Thus—to illustrate—in one exemplification, this invention provides novel TDCPs comprised of: any three members selected from at least three groups of ingredients as shown in Table 1; in another exemplification, this invention provides novel TDCPs comprised of: any four members selected from at least three groups of ingredients as shown in Table 1; in another exemplification, this invention provides novel TDCPs comprised of: any five members selected from at least three groups of ingredients as shown in Table 1; in another exemplification, this invention provides novel TDCPs comprised of: any six members selected from at least three groups of ingredients as shown in Table 1; (and so on); and in another exemplification, this invention provides novel TDCPs comprised of: any one thousand members selected from at least three groups of ingredients as shown in Table 1.

In separate but non-limiting exemplifications, this invention provides novel TDCPs comprised of: any “N4-1000” members selected from at least four groups of ingredients as shown in Table 1, where “N4-1000” is any integer from 4 up to 1000. Thus—to illustrate—in one exemplification, this invention provides novel TDCPs comprised of: any four members selected from at least four groups of ingredients as shown in Table 1; in another exemplification, this invention provides novel TDCPs comprised of: any five members selected from at least four groups of ingredients as shown in Table 1; in another exemplification, this invention provides novel TDCPs comprised of: any six members selected from at least four groups of ingredients as shown in Table 1; in another exemplification, this invention provides novel TDCPs comprised of: any seven members selected from at least four groups of ingredients as shown in Table 1; (and so on); and in another exemplification, this invention provides novel TDCPs comprised of: any one thousand members selected from at least four groups of ingredients as shown in Table 1.

In separate but non-limiting exemplifications, this invention provides novel TDCPs comprised of: any “N5-1000” members selected from at least five groups of ingredients as shown in Table 1, where “N5-1000” is any integer from 5 up to 1000. Thus—to illustrate—in one exemplification, this invention provides novel TDCPs comprised of: any five members selected from at least five groups of ingredients as shown in Table 1; in another exemplification, this invention provides novel TDCPs comprised of: any six members selected from at least five groups of ingredients as shown in Table 1; in another exemplification, this invention provides novel TDCPs comprised of: any seven members selected from at least five groups of ingredients as shown in Table 1; in another exemplification, this invention provides novel TDCPs comprised of: any eight members selected from at least five groups of ingredients as shown in Table 1; (and so on) . . . ; and in another exemplification, this invention provides novel TDCPs comprised of: any one thousand members selected from at least five groups of ingredients as shown in Table 1.

In separate but non-limiting exemplifications, this invention provides novel TDCPs comprised of: any “N6-1000” members selected from at least six groups of ingredients as shown in Table 1, where “N6-1000” is any integer from 6 up to 1000. Thus—to illustrate—in one exemplification, this invention provides novel TDCPs comprised of: any six members selected from at least six groups of ingredients as shown in Table 1; in another exemplification, this invention provides novel TDCPs comprised of: any seven members selected from at least six groups of ingredients as shown in Table 1; in another exemplification, this invention provides novel TDCPs comprised of: any eight members selected from at least six groups of ingredients as shown in Table 1; in another exemplification, this invention provides novel TDCPs comprised of: any nine members selected from at least six groups of ingredients as shown in Table 1; (and so on) . . . ; and in another exemplification, this invention provides novel TDCPs comprised of: any one thousand members selected from at least six groups of ingredients as shown in Table 1.

In separate but non-limiting exemplifications, this invention provides novel TDCPs comprised of: any “N7-1000” members selected from at least seven groups of ingredients as shown in Table 1, where “N7-1000” is any integer from 7 up to 1000. Thus—to illustrate—in one exemplification, this invention provides novel TDCPs comprised of: any seven members selected from at least seven groups of ingredients as shown in Table 1; in another exemplification, this invention provides novel TDCPs comprised of: any eight members selected from at least seven groups of ingredients as shown in Table 1; in another exemplification, this invention provides novel TDCPs comprised of: any nine members selected from at least seven groups of ingredients as shown in Table 1; in another exemplification, this invention provides novel TDCPs comprised of: any ten members selected from at least seven groups of ingredients as shown in Table 1; (and so on) . . . ; and in another exemplification, this invention provides novel TDCPs comprised of: any one thousand members selected from at least seven groups of ingredients as shown in Table 1.

The seven groups of ingredients in Table 1 are: Group 1, Group 2, Group 3, Group 4, Group 5, Group 6, and Group 7.

### Table 1: Exemplary Ingredients of Compositions of the Invention

TABLE 1
Exemplary Ingredients of compositions of the invention.

| Group | Group Members (Non-limiting examples are listed for each group) |
|---|---|
| 1 | Polar Solvents, Non-Polar Solvents, Inorganic Solvents, And Organic Solvents |
| | Water, methanol, ethanol, propanol (e.g. isopropanol), other alcohols, mixtures |
| | of water and one or more alcohol, DMSO, and other solvents. |
| 2 | Chelators |
| | 2,3-dimercaptopropanesulphonate sodium (2,3-dimercato-1-propanesulfonic |
| | acid, or DMPS), 2,3-dimercaptosuccinic acid (DMSA), British anti-Lewisite |
| | (BAL), other analogs of BAL, ethyleneglycol-bis[beta-aminoethyl ether]-N,N′- |
| | tetra-acetic acid (EGTA), ethyleneglycol-bis[beta-aminoethyl ether]-N,N′-tetra- |
| | acetic acid (EGTA), diethylenetriamine-pentaacetic acid (DTPA) and |
| | derivatives, dipropylenetriamine (DPTA), triethylenetetraaminehexaacetic acid |
| | (TTHA), N-hydroxyethylenediaminehexaacetic-acid (HEDHA), 1,4,7- |
| | triazacyclononane-N,N′,N″-triacetic acid (NOTA), 1,4,7,10- |
| | tetraazacyclododecane-N,N′,N″,N′′′-tetraacetic acid (DOTA), hydroxyethyl- |
| | ethylenediaminetriacetic acid (HEDTA, e.g., an N-(2-hydroxyethyl)- |
| | ethylenediaminetriacetic acid), other polyaminopolycarboxylic acids, |
| | iminodiacetic acid (IDA), cyclam, penicillamine, dimercaptosuccinic acid, |
| | tartrate, thiomalic acid, crown ethers, nitrilotriacetatic acid (NTA), 3,6- |
| | dioxaoctanedithioamide, 3,6-dioxaoctanediamide, salicyladoximine, dithio- |
| | oxamide, 8-hydroxyquinoline, cupferron, 2,2′-thiobis(ethyl acetoacetate), 2,2′- |
| | dipyridyl, and derivatives thereof (e.g., N,N′-bis-(pyridoxal-5-phosphate)- |
| | alkylenediamine-N,N′-diacetic acids, N,N′-bis-(pyridoxal-5-phosphate)-1,2- |
| | cycloalkylenediamine-N,N′-diacetic acids, and N,N′-bis-(pyridoxal-5- |
| | phosphate)-1,2-arylenediamine-N,N′-diacetic acids, the corresponding |
| | monophosphate compounds and monoacetic acid compounds, and their salts |
| | and esters), sodium citrate, or oxalate salts such as sodium, potassium, |
| | ammonium or lithium oxalate; including the disodium, trisodium, tetrasodium, |
| | dipotassium, tripotassium, dilithium and diammonium salts of EDTA; the |
| | barium, calcium, cobalt, copper, dysprosium, europium, iron, indium, |
| | lanthanum, magnesium, manganese, nickel, samarium, strontium, and zinc |
| | chelates of EDTA. Also included are any polyamino-polycarboxylic |
| | compounds, e.g., those derived from EDTA, DTPA, DOTA, TETA, TETRA, |
| | TITRA or 3,3,9,9-tetramethyl-4,8-diazaundecane-2,10-dione dioxime |
| | (HMPAO) or from such groups substituted, e.g. by a p-isothiocyanato- |
| | phenylC.sub.1-3 alkyl; and, oxime active agents, e.g. oximes such as |
| | bisquaternary and triquaternary oximes. |
| 3 | Sulfur-Containing Amino Acids, Sulfur-Containing Peptides, Sulfur-Containing |
| | Proteins, Antioxidant molecules (including, but non limited to, amino acids, |
| | peptides and proteins) |
| | Glutathione (GSH), methionine, cysteine, cystine (2 molecules of cysteine |
| | linked together), non-mammalian amino acids and proteins, other artificial or |
| | non-natural amino acids and proteins (e.g. man-made or artificially |
| | synthesized), agents for reducing oxidation (antioxidants, such as substituted |
| | phenols) and polymers of these (two or more of the aforementioned molecules |
| | linked together). Antioxidants include synthetic catalytic cyclic salen-metal |
| | antioxidants and reactive oxygen species scavengers. |
| 4 | Humectants and Thickeners |
| | Glycerin, polyethylene glycol, polypropylene glycol, other glycols, |
| | Colloid710h96, agar agar, carbomers (organic gelling agents), hydroscopic |
| | substituted cellulose, starches, propylene glycol glycerines, mono and poly |
| | glycols and sorbitols, and equivalent compounds, e.g., any compound that |
| | absorbs or retains moisture, including substances that preserve the moisture or |
| | water content of the skin, such as alpha hydroxy acids such as lactic acid. |
| 5 | Surfactants |
| | MJRY-52 (polyoxyethylene separate), other surfactants including anionic |
| | surfactants, quaternium cationic surfactants, and nonionic surfactants. |
| 6 | Creams, Lotions, Oils, and Waxes Containing: One Or More Phospholipids, |
| | Lipids or Fatty Acids |
| 7 | Lewis Acids and Lewis Bases |
| | Any Lewis acid or Lewis base, including for example, citric acid, acetic acid, |
| | niacin, and nicotinamide. |

### Exemplary Group 1 Members (e.g. Water)

Group 1 members include: water; methanol; ethanol; 1-propanol; 1-butanol; formic acid; acetic acid; formamide; acetone; methyl ethyl ketone; ethyl acetate; acetonitrile; N,N-dimethylformamide (DMF); dimethyl sulfoxide (DMSO); hexane; benzene; diethyl ether; tetrahydrofuran (THF); methylene chloride; and carbon tetrachloride.

### Exemplary Group 2 Members (e.g. DMPS, DTPA, EGTA, EDTA, BAL etc.)

Group 2 members include: chelators (i.e., chelating agents). Group 2 members include, but are not limited to: 2,3-dimercaptopropanesulphonate sodium (DMPS), meso-2,3-dimercaptosuccinic acid (DMSA), 1 Dimercaprol (BAL), analogs of BAL, EDTA, EGTA, dipropylenetriamine (DPTA), triethylenetetraaminehexaacetic acid (TTHA); N-hydroxyethylene-diaminehexaacetic-acid (HEDHA), 1,4,7-triazacyclononane-N,N′,N″-triacetic acid (NOTA); 1,4,7,10-tetraazacyclododecane-N,N′,N″,N′″-tetraacetic acid (DOTA), hydroxyethyl-ethylenediaminetriacetic acid (HEDTA, e.g., an N-(2-hydroxyethyl)-ethylenediaminetriacetic acid), other polyaminopolycarboxylic acids, iminodiacetic acid (IDA), cyclam, penicillamine, dimercaptosuccinic acid, tartrate, thiomalic acid, crown ethers, nitrilotriacetatic acid (NTA), 3,6-dioxaoctanedithioamide, 3,6-dioxaoctanediamide, salicyladoximine, dithio-oxamide, 8-hydroxyquinoline, cupferron, 2,2′-thiobis(ethyl acetoacetate), 2,2′-dipyridyl, and derivatives thereof, e.g., N,N′-bis-(pyridoxal-5-phosphate)-alkylenediamine-N,N′-diacetic acids, N,N′-bis-(pyridoxal-5-phosphate)-1,2-cycloalkylenediamine-N,N′-diacetic acids, and N,N′-bis-(pyridoxal-5-phosphate)-1,2-arylenediamine-N,N′-diacetic acids, the corresponding monophosphate compounds and monoacetic acid compounds, and their salts and esters, as described, e.g., in U.S. Pat. No. 4,992,555. According to this invention, other chelators that are members of Group 3 are provided herein or are otherwise known in the art and can serve as ingredients for this invention.

Additional exemplary chelating agents include diethylenetriamine-pentaacetic acid (DTPA) and derivatives, e.g., as described in U.S. Pat. No. 6,040,432 (e.g., N,N-bis-{2-[N′,N′-bis-(carboxymethyl)]-amino]-ethyl}-L-3-[(4-methoxy)-phenyl]-alanine), triethylene-tetraaminehexaacetic acid (TTHA), N-hydroxyethylene-diaminehexaacetic-acid (HEDHA), 1,4,7-triazacyclononane-N,N′,N″-triacetic acid (NOTA), 1,4,7,10-tetraazacyclododecane-N,N′,N″,N′″-tetraacetic acid (DOTA), and N′hydroxyethylenediamine-N,N,N′-triacetic acid (HEDTA), sodium citrate, or oxalate salts such as sodium, potassium, ammonium or lithium oxalate.

Additional exemplary chelating agents include iminodiacetic acid (IDA), cyclam, penicillamine, dimercaptosuccinic acid, tartrate, thiomalic acid, crown ethers, nitrilotriacetatic acid (NTA), 3,6-dioxaoctanedithioamide, 3,6-dioxaoctanediamide, salicyladoximine, dithio-oxamide, 8-hydroxyquinoline, cupferron, 2,2′-thiobis(ethyl acetoacetate), 2,2′-dipyridyl. IDA is a chelating headgroup which is selective for copper ions.

Additional exemplary chelators for use in the present invention also include, but are not limited to, ethylenediamine-N,N,N′,N′-tetraacetic acid (EDTA); the disodium, trisodium, tetrasodium, dipotassium, tripotassium, dilithium and diammonium salts of EDTA; the barium, calcium, cobalt, copper, dysprosium, europium, iron, indium, lanthanum, magnesium, manganese, nickel, samarium, strontium, and zinc chelates of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosphonic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11]pentatriacontane hexahydrobromide; and triethylenetetramine-N,N,N′,N″,N′″,N′″-hexaacetic acid. It is contemplated that any chelator which binds barium, calcium, cerium, cobalt, copper, iron, magnesium, manganese, nickel, strontium, or zinc will be acceptable for use in the present invention.

Additional exemplary chelators for use in conjunction with the present invention include ethylenediamine-N,N,N′,N′-tetraacetic acid (EDTA); the disodium, trisodium, tetrasodium, dipotassium, tripotassium, dilithium and diammonium salts of EDTA; 1,3-diamino-2-hydroxypropane-N,N,N′,N′-tetracetic acid; 1,3-diaminopropane-N,N,N′,N′-tetraacetic acid; O,O′-bis(2-aminoethyl)ethyleneglycol-N,N,N′,N′-tetraacetic acid; and 7,19,30-trioxa-1,4,10,13,16,22,27,33-octaazabicyclo[11,11,11]pentatriacontane hexahydrobromide.

In one aspect, the chelators for use in the present invention include ethylenediamine-N,N,N′,N′-tetraacetic acid (EDTA); the disodium salt of EDTA; 1,3-diaminopropane-N,N,N′,N′-tetraacetic acid; and O,O′-bis(2-aminoethyl)ethyleneglycol-N,N,N′,N′-tetraacetic acid.

In one aspect, a chelator may be selected from the group of chelators consisting of EDTA free acid, EDTA 2Na, EDTA 3Na, EDTA 4Na, EDTA 2K, EDTA 2Li, EDTA 2NH.sub.4, EDTA 3K, Ba(II)-EDTA, Ca(II)-EDTA, Co(II)-EDTA, Cu(II)-EDTA, Dy(HI)-EDTA, Eu(III)-EDTA, Fe(III)-EDTA, In(III)-EDTA, La(III)-EDTA, Mg(H)-EDTA, Mn(II)-EDTA, Ni(II)-EDTA, Sm(III)-EDTA, Sr(II)-EDTA, Zn(II)-EDTA, CyDTA, DHEG, DTPA-OH, DTPA, EDDA, EDDP, EDDPO, EDTA-OH, EDTPO, EGTA, HBED, HDTA, HIDA, IDA, Methyl-EDTA, NTA, NTP, NTPO, O-Bistren, and TTHA.

In one aspect, chelating groups include those derived from polyamino-polycarboxylic groups, e.g. those derived from EDTA, DTPA, DOTA, TETA, TETRA, TITRA or 3,3,9,9-tetramethyl-4,8-diazaundecane-2,10-dione dioxime (HMPAO) or from such groups substituted, e.g. by a p-isothiocyanato-phenylC.sub. 1-3 alkyl. In one aspect, p-isothiocyanatobenzyl is used. Chelating groups derived from DTPA can also be used.

In one aspect, chelating groups used in the compositions and methods of the invention include oxime active agents, e.g. oximes such as bisquaternary and triquaternary oximes, as described, e.g., in U.S. Pat. No. 5,902,816. In one aspect, chelating groups used in the compositions and methods of the invention include multiply substituted DTPA derivatives as described, e.g., in U.S. Pat. No. 5,885,548.

3,6,9-triaza-3,9-bis-(carboxymethyl)-6-{2->4-(1,4,7-trioxaoctyl)-phenyl!-1-carboxyethyl}-4,8-bis->4-(1,4,7-trioxaoctyl)-benzyl-undecanedioic acid or a salt thereof with physiologically acceptable cations; e.g., 3. 6,9-triaza-3,6,9-tris-(carboxymethyl)-4,8-bis-(4-ethoxybenzyl)-undecanedioic acid or a salt thereof with physiologically acceptable cations; 3,6,9-triaza-3,6,9-tris-(carboxymethyl)-2,10-bis-(4-ethoxybenzyl)-undecaned ioic acid or a salt thereof with physiologically acceptable cations, and the like.

In one aspect, the compositions of the invention comprise a TDCP, wherein the chelating group is derived from ethylene diaminetetraacetic acid (EDTA), diethylene triamine pentaacetic acid (DTPA), ethylene glycol-O,O′-bis(2-aminoethyl)-N,N,N′,N′-tetraacetic acid (EGTA), N,N′-bis(hydroxybenzyl)ethylenediamine-N,N′-diacetic acid (HBED), triethylenetetramine hexaacetic acid (TTHA), substituted EDTA or -DTPA 1,4,7,10-tetra-azacyclododecane-N,N′,N″,N′″-tetraacetic acid (DOTA) and 1,4,8,11-tetraazacyclotetradecane-N,N′,N″,N′″-tetraacetic acid (TETA), in free form or in pharmaceutically accepted salt form.

In one aspect, this invention provides a TDCP, wherein the chelating group is derived from 1,4,7,10-tetraazacyclotridecane-1,4,7,10-tetraacetic acid (TITRA), 1,4,8,11-tetraazacyclotetradecane (TETRA); EDTA, DTPA, DOTA, TETA, TITRA, TETRA or 3,3,9,9-tetramethyl-4,8-diazaundecane-2,10-dione dioxime (HMPAO) substituted by p-isothiocyanato-phenyl-C.sub.1-3 allyl, in free form or in pharmaceutically accepted salt form.

### Exemplary Group 3 Members (e.g. Glutathione, Antioxidants)

Group 3 embers include: sulfur-containing amino acids, sulfur-containing peptides, sulfur-containing proteins, and other sulfur-containing substances. Antioxidants used in the formulations and preparations of the invention include synthetic catalytic cyclic salen-metal antioxidants and reactive oxygen species scavengers, e.g., as described in U.S. Pat. No. 6,589,948.

### Exemplary Group 4 Members (e.g. Glycerin)

Group 4 embers include: glycerin, polyethylene glycol, polypropylene glycol, other glycols, Colloid710h96, agar agar, carbomers (organic gelling agents), hydroscopic substituted cellulose, and starches.

### Exemplary Group 5 Members (e.g. MJRY-52)

Group 5 embers include: MJRY-52 (polyoxyethylene separate), other surfactants including anionic surfactants and nonionic surfactants. Anionic surfactants include, by way of non-limiting exemplification: alkyldiphenyloxide disulfonate salts; dioctyl sulfosuccinates; phosphate esters; sulfates and sulfonates. Non-ionic surfactants include, by way of non-limiting exemplification: alkyl amine ethoxylates; alkyl polyglucosides; branched secondary; alcohol ethoxylates; ethylene oxide/propylene oxide copolymers; low foam surfactants; nonylphenol ethoxylates; octylphenol ethoxylates; secondary alcohol ethoxylates; specialty alkoxylates.

### Exemplary Group 6 Members (e.g. Lecithin)

Group 6 members include creams lotions oils and waxes containing: one or more phospholipids, lipids or fatty acids. Additional exemplary group 6 members include phospholipids, lipids and fatty acid members including, but not limited to: lecithin, phosphatidylcholine, phosphatidylserine, phosphatidylethanolamine, dilinoleylphosphatidylcholine, lysolipids, dipalmitoylphosphatidylcholine, distearoylphosphatidylcholine, phosphatidylcholine, phosphatidic acid, sphingomyelin, cholesterol, cholesterol sulfate, cholesterol hemisuccinate, tocopherol hemisuccinate, phosphatidylethanolamine, phosphatidylinositol, ferulic acid, fatty acids (e.g. palmitic acid, stearic acid, oleic acid, linolenic acid, linoleic acid, etc.), glycosphingolipids, glucolipids, glycolipids, sulphatides, lipids bearing sulfonated mono-, di-, oligo- or polysaccharides, lipids with ether and ester-linked fatty acids, triglycerides, lipoproteins (high or low density), lipids and polymerized lipids; animal and vegetable oils, hydrocarbon oils, ester oils, silicone oils, higher fatty acids, higher alcohols, sunscreening agents, and flavors (e.g. oily flavors).

### Exemplary Group 7 Members (e.g. Citric Acid)

Group 1 members include: water; methanol; ethanol; 1-propanol; 1-butanol; formic acid; acetic acid; formamide; acetone; methyl ethyl ketone; ethyl acetate; acetonitrile; N,N-dimethylformamide (DMF); dimethyl sulfoxide (DMSO); hexane; benzene; diethyl ether; tetrahydrofuran (THF); methylene chloride; and carbon tetrachloride.

The invention also relates to transdermal delivery of active agents, chelators, pharmaceuticals and co-enzymes (B1, B2, B3, B5, B6, B7, B9, H and the like) across the epidermal (skin) barrier of humans and mammals. In one aspect, the invention provides methods for developing new transdermal delivery systems, e.g., for any polar or non-polar active agent of small or large molecular size. These delivery systems can rapidly delivering the active agent to a targeted location systemically or locally.

Many methods for making preparations and formulations of the invention comprising the ingredients provided herein as well as many methods for making preparations comprising the ingredients described herein are in the art. These preparation protocols are well known in the art, e.g., as described by references regarding these methods (which are hereby incorporated by reference in their entirety, see statement above), e.g., Patricia A. L. Kongshavn: The Science of Glutathione; Lomaestro B, Malone M. Glutathione in health and disease: Pharmacotherapeutic Issues Ann Pharmacother 29: 1263-73,1995; Patricia A. L. Kongshavn: Glutathione the undiscovered “natural dog”; Lands L C, Grey V L, Smountas AA: Effect of supplementation with a cysteine donor on muscular performance. J Appl Physiol 87:1381-5, 1999.

The pharmaceutical compositions and preparations of the invention can be formulated in any way and can be administered in a variety of unit dosage forms depending upon the particular drug (e.g., chelator), medicine, vitamin, natural products or other composition to be delivered, the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”). Pharmaceutical compositions, formulations and preparations of the invention can be prepared according to any method known to the art for the manufacture of pharmaceuticals. For example, pharmaceutical compositions, formulations and preparations of the invention can be prepared as described e.g., in U.S. Pat. No. 6,444,234; or, U.S. Pat. No. 6,787,152.

For example, pharmaceutical compositions, formulations and preparations of the invention can comprise a non-aqueous non-toxic solvent, such as a lower aliphatic mono- and poly-hydroxy compounds; a limonene, lemon oil or mixture of limonene and lemon oil; a methylsulfonylmethane; a skin stabilizer (e.g., aliphatic carboxylic acids, an aliphatic alcohols, Vitamin D3); a solute modifier (e.g., 3,3′-thiodipropionic acid, an ester thereof, or a salt thereof; an oxindole alkaloid, a polyphenolic flavonoid, a sugar adduct of a gluconuide, isoflavones, phosphatidyl serine, phosphatidyl choline, Vitamin D3 and Vitamin K1; and/or adenosine triphosphate (ATP) or a compound which induces generation of cyclic adenosine 3′5′ monophosphate cAMP iii situ or cGMP in situ, as described in U.S. Pat. No. 6,444,234. Solvents or solvent modifiers (e.g., lower alcohols, such as from about 2 to about 6 carbon atoms, monoalcohols such as ethanol, isopropanol, sec-butanol, polyols such as ethylene glycol, propylene glycol, butylene glycol, glycerol, ketone, acetone, methylethyl ketone, ethers, ethylether, mixtures thereof) in amounts which will be safe and non-toxic in use can be used in the pharmaceutical compositions, formulations and preparations of the invention as described in U.S. Pat. No. 6,787,152. Solvent modifiers (e.g., lemon oil, d-limonene, Vitamin E, pro-vitamin B, D-panthenol, methylsulfonylmethane (MSM)) can also be used. Pharmaceutical compositions, formulations and preparations of the invention can be prepared as described in U.S. Pat. Nos. 4,879,275; 3,996,934; and 3,731,683.

Pharmaceutical formulations and preparations of the invention also can comprise preserving agents. A formulation can be admixtured with nontoxic pharmaceutically acceptable excipients which are suitable for manufacture.

The invention will be further described with reference to the following examples; however, it is to be understood that the invention is not limited to such examples.

### EXAMPLES

### Example 1

### Transdermal Chelation Preparation (TDCP)

EXAMPLE 1
Transdermal Chelation Preparation (TDCP)

| % Weight | Ingredient | Conc. |
|---|---|---|
| 83.12% | WATER | |
| 3.93% | DMPS | 1 mg/drop |
| 11.94% | GLUTATHIONE | |
| 3.25% | GLYCERIN | |
| 3.25% | POLYETHYLENE GLYCOL | |
| 0.65% | MJRY50 | |
| 10.39% | CREAM | |
| 0.26% | CITRIC ACID | |
| 0.14% | COLLOID710H96 | |

In one aspect, DMPS ranges from 0.001 to 4.2 mg/drop; e.g. 0.1 mg per drop. Additional exemplary humectants, wetting agents and thickeners include those members of this group that also have anti-microbial effects; e.g. propylene glycol is a wetting agent for the colloids, a humectant for the skin, and an anti-microbial in solution.

Humectants maintain epidermal hydration to permit DMPS delta time penetration. There are numerous other glycols and humectants which can be substituted.

Citric acid is a Lewis acid for conjugation with the Lewis base DMPS. They do not react covalently or do so to a small extent, rather the majority of the bonds formed are non-covalent. Niacin and niocinamide can also be used, however, children's skin may be more sensitive than adult skin, and some children may be sensitive and develop a rash. Nevertheless, it does work. Any non-reactive Lewis acid or base could be utilized: acetic acid, citric acid, etc. Citric acid is suitable because it is compatible with the biological system.

An exemplary cream is composed of:

| | Weight percent | |
|---|---|---|
| Oils | 25% | ACTIVE |
| Stearyl Alcohol | 25% | INACTIVE |
| Oleyl Alcohol | 2% | INACTIVE |
| Lecithin | 1% | PHOSPHOLIPID for colloid |
| | | preparation. |
| Propylene Glycol | 11% | HUMECTANT & |
| | | ANTI-MICROBIAL |
| MyrJ-52 | 4% | SURFACTANT |
| Sorbitol | 1% | HUMECTANT |
| Methyl Paraben | 0.4% | Preservative |
| Propyl Paraben | 0.2% | PRESERVATIVE |
| Distilled water | 30.4% | |

Any Phospholipid could be employed to mfg a cream. Lecithin is suitable, because it is natural to or compatible with biological systems. Acetyl choline is an example of another suitable phospholipid.

Stearyl alcohol, oleyl alcohol and lecithin acts as barriers to loss of humectant activity and the related loss of DMPS and/or oxidation. This increases the available time for DMPS to transit the skin.

The following exemplary oils are chosen for Example 1. They are metabolic active and act both as transporter agents of DMPS and as protective agents from oxidation and to maintain the humectants in the skin. Any range of concentrations is active and can be used; the appropriate concentration for any particular oils (or any other ingredient in a formulation of the invention) for a particular purpose or indication can be determined by routine screening.

| | |
|---|---|
| 46.96% | Lauric acid |
| 19.57% | Myristic acid |
| 8.80% | Caprylic acid |
| 7.83% | Palmetic acid |
| 6.85% | Capric acid |
| 6.52% | Oleic acid |
| 1.09% | Palmitic acid |
| 0.98% | Linolenic acid |
| 0.43% | Steareic acid |
| 0.43% | Linoleic acid |
| 0.33% | Palmitoleic acid |
| 0.11% | Squalene |
| 0.11% | Tocopherols a, b, g, d |

### Example 2

### Determining the Activity and Level of Activity of the Chelator(s)

This example describes methods for determining the activity and level of activity of the chelator(s) used in the preparations and formulations of the invention.

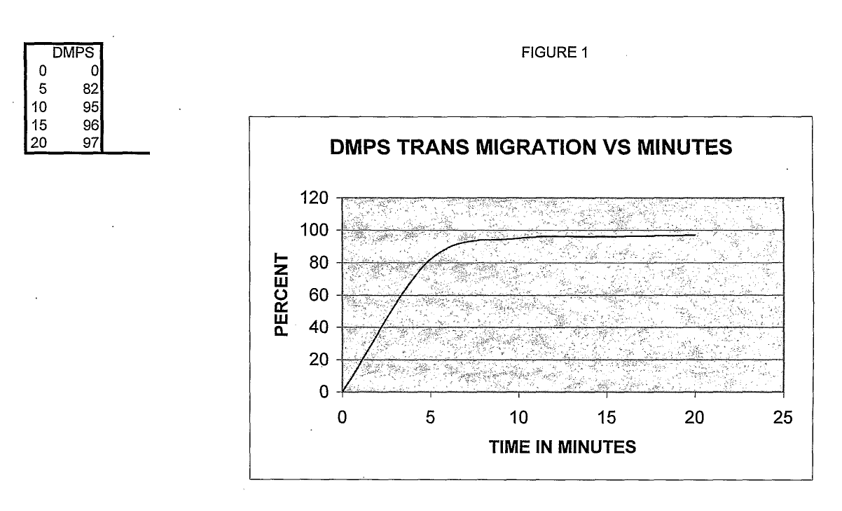

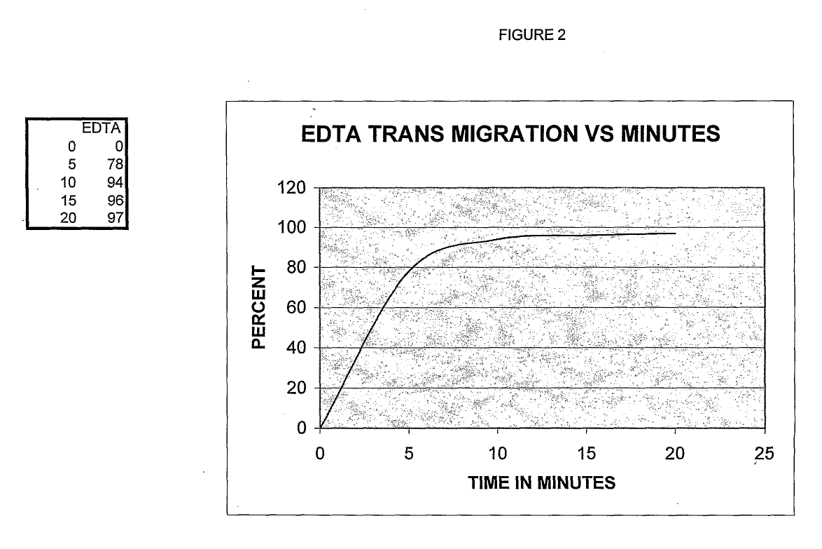

A fresh copper surface is prepared. The surface is gently heat oxidized. Next, the chelation quality was determined. About 0.0001 grams per drop (or 0.0029 g/ml) chelator effectively removed all the oxide form 1 cm sq in two minutes at a temperature of 98-99 degrees F. Then, the solution was diluted at 10% intervals to determine the point where it would not remove the oxide. There was a step wise decrease in chelation effect across the Cu test sample plate. A standard of chelation was graphed, see FIGS. 1 and 2.

A grid 1 sq cm area of an arm was marked off and the chelator solution applied to the skin; samples were taken at designated intervals. It was required to determine the correlation of one sq cm of skin and the application rate per one sq cm., in order to correlate it with the standard solution.

The samples were taken with a tungsten carbide industrial fiber tow cutter blade, which was aged in the chelating solution (to saturate the metal with chelator), and washed gently with distilled water.

A sq cm area of the applied solution was gently removed (without cutting the skin—without removing the stratum corneum). The sample was tested against the standard, and the approximate active quantity of chelator was determined. The sample was taken and restored in water with a dilution factor and tested it on the Cu for comparison. Multiple samples were taken and analyzed to determine the activity.

A similar test is used for thermal stability and activity. The chelators DMPS and EDTA are 100% inactive at 143 degrees F. for 5 minutes. Also, the identical test is used to determine batch activity. A crude field test is to put the chelator on a penny and heat to 98 degrees.

The invention will be further described with reference to the following examples; however, it is to be understood that the invention is not limited to such examples.

While the invention has been described in detail with reference to certain Exemplary aspects thereof, it will be understood that modifications and variations are within the spirit and scope of that which is described and claimed.

1: A preparation or formulation for topical or transdermal application comprising:
(a) at least two members, wherein the at least two members are selected from at least two groups of ingredients as set forth in Table 1;
(b) the preparation or formulation of (a), wherein the preparation or formulation comprises: at least one member selected from the group consisting of at least one solvent; and, at least one metal chelator;
(c) the preparation or formulation of (a), wherein the solvent comprises a polar solvent, a non-polar solvent, an inorganic solvent or an organic solvent;
(d) the preparation or formulation of (c), wherein the solvent comprises a water, an alcohol, a mixture of water and one or more alcohols, and dimethyl sulfoxide (DMSO);
(e) the preparation or formulation of (a) or (b), wherein the at least one metal chelator is selected from the group consisting of: 2,3-dimercaptopropanesulphonate sodium (2,3-dimercato-1-propanesulfonic acid, or DMPS); 2,3-dimercaptosuccinic acid (DMSA); British anti-Lewisite (BAL); analogs of BAL; calcium disodium ethylenediaminetetraacetate (EDTA); ethyleneglycol-bis[beta-aminoethyl ether]-N,N′-tetra-acetic acid (EGTA); diethylenetriamine-pentaacetic acid (DTPA); dipropylenetriamine (DPTA); triethylenetetraaminehexaacetic acid (TTHA); N-hydroxyethylene-diaminehexaacetic-acid (HEDHA); 1,4,7-triazacyclononane-N,N′,N″-triacetic acid (NOTA); 1,4,7,10-tetraazacyclododecane-N,N′,N″,N′″-tetraacetic acid (DOTA); HEDTA; a polyaminopolycarboxylic acid; iminodiacetic acid (IDA); cyclam; penicillamine; dimercaptosuccinic acid; tartrate; thiomalic acid; a crown ether; nitrilotriacetatic acid (NTA); 3,6-dioxaoctanedithioamide; 3,6-dioxaoctanediamide; salicyladoximine; dithio-oxamide; 8-hydroxyquinoline; cupferron; 2,2′-thiobis(ethyl acetoacetate); 2,2′-dipyridyl and derivatives thereof; sodium citrate; and, an oxalate salt;
(f) the preparation or formulation of (e), wherein the oxalate salt comprises a sodium oxalate salt, potassium oxalate salt, ammonium oxalate salt or lithium oxalate salt; alternatively the EDTA comprises disodium, trisodium, tetrasodium, dipotassium, tripotassium, dilithium or diammonium salts of EDTA; alternatively the EDTA comprises the barium, calcium, cobalt, copper, dysprosium, europium, iron, indium, lanthanum, magnesium, manganese, nickel, samarium, strontium or zinc chelates of EDTA;
(g) the preparation or formulation of (d), wherein the at least one alcohol is selected from the group consisting of a methanol, an ethanol, a propanol and an isopropanol;
(h) the preparation or formulation of (a), wherein the preparation or formulation comprises: at least one solvent; and, at least one member selected from the group consisting of a sulfur-containing amino acid, a sulfur-containing peptide, a sulfur-containing protein and an antioxidant molecule;
(i) the preparation or formulation of (h), wherein the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO);
(j) the preparation or formulation of (h), wherein the at least one member selected from the group consisting of a sulfur-containing amino acid, a sulfur-containing peptide, a sulfur-containing protein and an antioxidant molecule comprises a glutathione (GSH), a methionine, a cysteine or a cystine;
(k) the preparation or formulation of (a), wherein the preparation or formulation comprises: at least one solvent; and, a humectant or a thickener;
(l) the preparation or formulation of (k), wherein the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO);
(m) the preparation or formulation of (k), wherein the humectant or thickener comprises a glycerin, a polyethylene glycol, a polypropylene glycol, a glycol, a colloid710h96, an agar agar, a carbomer, a hydroscopic substituted cellulose, a starch, a propylene glycol glycerine, a mono and poly glycols or a sorbitol;
(n) the preparation or formulation of (a), wherein the preparation or formulation comprises: at least one solvent; and, a surfactant;
(o) the preparation or formulation of (n), wherein the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO);
(p) the preparation or formulation of (n), wherein the surfactant comprises a MJR-52 (polyoxyethylene separate), an anionic surfactant, a quaternium cationic surfactant or a nonionic surfactant;
(q) the preparation or formulation of (a), wherein the preparation or formulation comprises: at least one solvent; and, a cream, a lotion, an oil, a wax and a wax comprising at least one phospholipid, lipid or fatty acid;
(r) the preparation or formulation of (q), wherein the at least one phospholipid, lipid or fatty acid comprises a lecithin, a phosphatidylcholine, a phosphatidylserine, a phosphatidylethanolamine, a dilinoleylphosphatidylcholine, a lysolipid, a dipalmitoylphosphatidylcholine, a distearoylphosphatidylcholine, a phosphatidylcholine, a phosphatidic acid, a sphingomyelin, a cholesterol, a cholesterol sulfate, a cholesterol hemisuccinate, a tocopherol hemisuccinate, a phosphatidylethanolamine, a phosphatidylinositol, a ferulic acid, a fatty acid, a palmitic acid, a stearic acid, an oleic acid, a linolenic acid, a linoleic acid, a glycosphingolipid, a glucolipid, a glycolipid, a sulphatide, a lipid comprising sulfonated mono-, di-, oligo- or polysaccharides, a lipid comprising an ether or an ester-linked fatty acid, a triglyceride, a high density lipoprotein, a low density lipoprotein, a polymerized lipid, an animal or vegetable oil, a hydrocarbon oil, an ester oil, a silicone oil, a higher fatty acid, a higher alcohol, a sunscreening agent or a flavor;
(s) the preparation or formulation of (n), wherein the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO);
(t) the preparation or formulation of (a), wherein the preparation or formulation comprises: at least one solvent; and, a Lewis acid or Lewis base;
(u) the preparation or formulation of (t), wherein the solvent comprises at least one member selected from the group consisting of a polar solvent, a non-polar solvent, an inorganic solvent or an organic solvent, wherein alternatively the solvent comprises water, an alcohol, a mixture of water and one or more alcohols, or dimethyl sulfoxide (DMSO);
(v) the preparation or formulation of (t), wherein the Lewis acid or Lewis base comprises a citric acid, an acetic acid, a niacin or a nicotinamide;
(w) the preparation or formulation of (a), wherein the preparation or formulation comprises: at least one metal chelator; and, at least one member selected from the group consisting of a sulfur-containing amino acid, a sulfur-containing peptide, a sulfur-containing protein and an antioxidant molecule, wherein alternatively the sulfur-containing amino acid, sulfur-containing peptide, sulfur-containing protein or antioxidant molecule comprises a glutathione (GSH), a methionine, a cysteine or a cystine;
(x) the preparation or formulation of (a), wherein the preparation or formulation comprises: at least one metal chelator; and, a humectant or a thickener; or, the preparation or formulation comprises: at least one metal chelator; and, a surfactant; or, the preparation or formulation comprises: at least one metal chelator; and, a cream, a lotion, an oil, a wax and a wax comprising at least one phospholipid, lipid or fatty acid; or the preparation or formulation comprises: at least one metal chelator; and, a Lewis acid or Lewis base, wherein alternatively the Lewis acid or Lewis base comprises a citric acid, an acetic acid, a niacin or a nicotinamide;
(y) the preparation or formulation of any of (a) to (x), wherein the at least one metal chelator is selected from the group consisting of: 2,3-dimercaptopropanesulphonate sodium (2,3-dimercato-1-propanesulfonic acid, or DMPS); 2,3-dimercaptosuccinic acid (DMSA); British anti-Lewisite (BAL); analogs of BAL; calcium disodium ethylenediaminetetraacetate (EDTA); ethyleneglycol-bis[beta-aminoethyl ether]-N,N′-tetra-acetic acid (EGTA); diethylenetriamine-pentaacetic acid (DTPA); dipropylenetriamine (DPTA); triethylenetetraaminehexaacetic acid (TTHA); N-hydroxyethylenediaminehexaacetic-acid (HEDHA); 1,4,7-triazacyclononane-N,N′,N″-triacetic acid (NOTA); 1,4,7,10-tetraazacyclododecane-N,N′,N″,N′″-tetraacetic acid (DOTA); HEDTA; a polyaminopolycarboxylic acid; iminodiacetic acid (IDA); cyclam; penicillamine; dimercaptosuccinic acid; tartrate; thiomalic acid; a crown ether; nitrilotriacetatic acid (NTA); 3,6-dioxaoctanedithioamide; 3,6-dioxaoctanediamide; salicyladoximine; dithio-oxamide; 8-hydroxyquinoline; cupferron; 2,2′-thiobis(ethyl acetoacetate); 2,2′-dipyridyl and derivatives thereof; sodium citrate; and, an oxalate salt; or the oxalate salt comprises a sodium oxalate salt, potassium oxalate salt, ammonium oxalate salt or lithium oxalate salt; alternatively the EDTA comprises disodium, trisodium, tetrasodium, dipotassium, tripotassium, dilithium or diammonium salts of EDTA; alternatively the EDTA comprises the barium, calcium, cobalt, copper, dysprosium, europium, iron, indium, lanthanum, magnesium, manganese, nickel, samarium, strontium or zinc chelates of EDTA; or
(z) the preparation or formulation of (a), wherein the preparation or formulation comprises: at least one member selected from the group consisting of a sulfur-containing amino acid, a sulfur-containing peptide, a sulfur-containing protein and an antioxidant molecule; and, a humectant or a thickener; or the preparation or formulation comprises: at least one member selected from the group consisting of a sulfur-containing amino acid, a sulfur-containing peptide, a sulfur-containing protein and an antioxidant molecule, and, a surfactant; or the preparation or formulation comprises: at least one member selected from the group consisting of a sulfur-containing amino acid, a sulfur-containing peptide, a sulfur-containing protein and an antioxidant molecule; and, a cream, a lotion, an oil, a wax and a wax comprising at least one phospholipid, lipid or fatty acid; or the preparation or formulation comprises: at least one member selected from the group consisting of a sulfur-containing amino acid, a sulfur-containing peptide, a sulfur-containing protein and an antioxidant molecule; and, a Lewis acid or Lewis base, or the Lewis acid or Lewis base comprises a citric acid, an acetic acid, a niacin or a nicotinamide; or wherein the sulfur-containing amino acid, sulfur-containing peptide, sulfur-containing protein or antioxidant molecule comprises a glutathione (GSH), a methionine, a cysteine or a cystine; or wherein the preparation or formulation comprises: at least one humectant or thickener; and, a surfactant; or wherein the preparation or formulation comprises: at least one humectant or thickener; and, a cream, a lotion, an oil, a wax and a wax comprising at least one phospholipid, lipid or fatty acid.

2-33. (canceled)

34: The preparation or formulation of claim 1, wherein the preparation or formulation comprises:
(a) at least one humectant or thickener; and, a Lewis acid or Lewis base, wherein alternatively the Lewis acid or Lewis base comprises a citric acid, an acetic acid, a niacin or a nicotinamide;
(b) the preparation or formulation of (a), wherein the humectant or thickener comprises a glycerin, a polyethylene glycol, a polypropylene glycol, a glycol, a colloid710h96, an agar agar, a carbomer, a hydroscopic substituted cellulose, a starch, a propylene glycol glycerine, a mono and poly glycols or a sorbitol;
(c) the preparation or formulation of (a), wherein the preparation or formulation comprises: at least one surfactant; and, a cream, a lotion, an oil, a wax and a wax comprising at least one phospholipid, lipid or fatty acid;
(d) the preparation or formulation of (a), wherein the preparation or formulation comprises: at least one surfactant; and, a Lewis acid or Lewis base, wherein alternatively the Lewis acid or Lewis base comprises a citric acid, an acetic acid, a niacin or a nicotinamide;
(e) the preparation or formulation of (a), (b) or (c), wherein the at least one surfactant comprises a MJRY-52 (polyoxyethylene separate), an anionic surfactant, a quaternium cationic surfactant or a nonionic surfactant;
(f) the preparation or formulation of (a), wherein the preparation or formulation comprises: at least a cream, a lotion, an oil, a wax or a wax comprising at least one phospholipid, lipid or fatty acid; and, a Lewis acid or Lewis base;
(g) the preparation or formulation of (f), wherein Lewis acid or Lewis base comprises a citric acid, an acetic acid, a niacin or a nicotinamide;
(h) the preparation or formulation of (a), wherein the preparation or formulation further comprises a compound that alters intracellular osmotic pressure, wherein alternatively the compound comprises niacin, niacinamide, vitamin B3, B5 or B12 and/or biotin;
(i) the preparation or formulation of (a), wherein the preparation or formulation is formulated as a lotion, a cream, a milk, an emulsion, a powder, a spray, an aerosol, a gel or a patch-associated composition;
(j) the preparation or formulation of (a), further comprising a drug, a medicament, a vitamin, a co-enzyme, or a natural product or a combination thereof;
(k) the preparation or formulation of (a), wherein the preparation or formulation is formulated with a drug, a medicament, a vitamin, a co-enzyme, or a natural product at an effective therapeutic dose of less than about 1 mg per day, or, alternatively, 2, 3, 4, 5, 6, 7, 8, 9, 10 or more mg per day, or, alternatively, 0.9, 0.8, 0.7, 0.6, 0.5, 0.4, 0.3, 0.2 or 0.1 mg per day, or less;
(l) the preparation or formulation of (a), wherein the preparation or formulation comprises molecules that can modify or ameliorate an immune response, wherein alternatively the altered immune response is an altered humoral or cellular response;
(m) the preparation or formulation of (a), wherein the drug, medicament, vitamin, coenzyme or natural product has a molecular weight (MW) less than about 340 daltons; or
(n) the preparation or formulation of (a), formulated such that the drug, medicament, vitamin, coenzyme or natural product is delivered in amounts less than about 1.0, 2, 3, 4, 5, 6, 7, 8, 9 or 10 or more mg per 24 hours.

35-47. (canceled)

48: A method of making a specific transdermal delivery system comprising:
(a) selecting one or more active agents, wherein the active agent comprises a chelator;
(b) determining a transdermal effective dose of the active agent:
(c) quantifying of effective dose of the active agent;
(d) determining an amount of a solvent to solubilize the effective dose of active agent; and,
(e) quantifying amount of solvent to solubilize said effective dose.

49: A method of preventing or ameliorating a disease or condition mediated or caused by a heavy metal comprising:
(a) providing the preparation or formulation claim 1; and
(b) administering by topical or transdermal application the preparation or formulation of step (a) in an amount effective to prevent or ameliorate the disease or condition.

50: A method for detoxifying an individual of a heavy metal comprising:
(a) providing the preparation or formulation in claim 1; and
(b) administering by topical or transdermal application the preparation or formulation of step (a) in an amount effective to detoxify the individual.

51: A lotion, a cream, a milk, an emulsion, a powder, a spray, an aerosol, a gel or a patch comprising
(a) the preparation or formulation claim 1;
(b) the lotion, cream, milk, emulsion, powder, spray, aerosol, gel or patch of (a), further comprising a drug, a medicament, a vitamin, a coenzyme or a natural product; or
(c) the lotion, cream, milk, emulsion, powder, spray, aerosol, gel or patch of (a), wherein the preparation or formulation is formulated such that the drug, medicament, vitamin, coenzyme or natural product is delivered in amounts less than about 1.0, 2, 3, 4, 5, 6, 7, 8, 9 or 10 or more mg per 24 hours.

52-53. (canceled)

54: A preparation or formulation for transdermal or transepithelial delivery of a composition comprising (a) a 2-3, dimercaptopropane-1-sulfonate (DMPS) or a glutathione formulated in a stabilizing base, or (b) the preparation or formulation of (a) wherein the preparation or formulation further comprises a methionine.

55: lotion, a cream, a milk, an emulsion, a powder, a spray, an aerosol, a gel or a patch comprising a 2-3, dimercaptopropane-1-sulfonate (DMPS) or a glutathione, wherein alternatively the preparation or formulation further comprises a methionine.

56: A preparation or formulation for transdermal or transepithelial delivery of a composition comprising water, DMPS or glutathione, a glycerin, a polyethylene glycol, MJR50, a cream and citric acid.

57: A lotion, a cream, a milk, an emulsion, a powder, a spray, an aerosol, a gel or a patch comprising water, DMPS or glutathione, a glycerin, a polyethylene glycol, MJRY50, a cream and a citric acid.

58: A preparation or formulation comprising water, a glutathione, a glycerin, a polyethylene glycol, MJRY50 and a cream.

59: A lotion, a cream, a milk, an emulsion, a powder, a spray, an aerosol, a gel or a patch comprising water, glutathione, glycerin, polyethylene glycol, MJRY50 and a cream.

60: A lotion or cream comprising (a) lecithin, propylene glycol, a surfactant and a sorbitol, or (b) the lotion or cream of (a), wherein the surfactant is MyrJ-52.

61: A pharmaceutical composition comprising a preparation or formulation claim 1.

62: A lotion, a cream, a milk, an emulsion, a powder, a spray, an aerosol, a gel or a patch comprising a preparation or formulation of claim 1, and Vitamin A or beta-carotene, Vitamin B1 (as Thiamin or Thiamin mononitrate), Vitamin B (as riboflavin), Vitamin B3 (as Niacin), Vitamin B6 (as Pyridoxine or Pyridoxine hydrochloride), Vitamin B9 (Folic Acid), Vitamin B12 (cyanocobalamine or hydroxycobalamine), Vitamin H (biotin), Vitamin C (ascorbic acid), Vitamin D, Vitamin E (as dl-alpha acetate), Vitamin K, dexpanthenol, nicotinamide (niacinamide), tocopheryl, or a mixture thereof.
